(19) United States
(12) Patent Application Publication
(10) Pub. No.: US 2012/0265047 A1
(43) Pub. Date: Oct. 18, 2012

(54) INTRAOCULAR PRESSURE DETECTING DEVICE AND DETECTING METHOD THEREOF

(75) Inventors: MENG-SHIN YEN, Taipei City (TW); William Wang, Taoyuan City (TW); Chung-Cheng Chou, Taoyuan County (TW)

(73) Assignee: Crystalvue Medical Corporation

(21) Appl. No.: 13/446,168

(22) Filed: Apr. 13, 2012

(30) Foreign Application Priority Data
Apr. 14, 2011 (TW) 100112920

(51) Int. Cl. A61B 3/16

(52) U.S. Cl. 600/405

(57) ABSTRACT

An intraocular pressure detecting device includes an optical module and a data processing unit. The light module transmits a light beam to an eyeball and acquires at least one light interference signal of a reflected light beam reflected from a cornea and a reference light beam. The light module electrically couples with the data processing unit, which determines an intraocular pressure detection area according to the at least one light interference signal. The data processing unit utilizes the at least one light interference signal acquired by the light module to calculate the intraocular pressure.

### BACKGROUND OF THE INVENTION

1. Field of the Invention

The present invention generally relates to an intraocular pressure detecting device and a detecting method thereof. Particularly, the present invention relates to an intraocular pressure detecting device capable of determining an intraocular pressure detection area and a detecting method thereof.

2. Description of the Prior Art

The conventional intraocular pressure detecting device for measuring and controlling the relative fluid pressure inside the eye usually includes a surgical operation appliance invasive to the eye. A fluid pressure converter is disposed on the surgical operation appliance; when the invasive appliance is applied to the eye, the converter is positioned near an opening. The opening communicates with the interior of the eye so that the converter can reflect the change in fluid pressure and generate a signal in response to the change in fluid pressure. In other words, the conventional intraocular pressure detecting device is invasive to the eye when measuring the intraocular pressure. Accordingly, it is difficult for the public to accept the invasive type intraocular pressure detecting device.

The invasive type intraocular pressure detecting device is gradually replaced by modern intraocular pressure detecting devices. The non-invasive intraocular pressure detecting devices can be classified into contact type and non-contact type. Either the contact type or the non-contact type intraocular pressure detecting devices requires imposing a force on the cornea, and the value of eye pressure is then deduced from the relationship between the force and the deformation of cornea. However, the actual measurement reveals that the corneal curvature and the corneal thickness of the intraocular pressure detection area influence the eye pressure measurement and a deviation of measurement value exits. Accordingly, an intraocular pressure detecting device capable of determining a proper intraocular pressure detection are is a key research approach for the industry.

### SUMMARY OF THE INVENTION

It is an object of the present invention to provide an intraocular pressure detecting device and an intraocular pressure detecting method that can determine an appropriate intraocular pressure detection area.

In order to achieve the above-mentioned object, the present invention provides an intraocular pressure detecting device, which includes an optical module and a data processing unit. The optical module transmits a light beam to an eyeball and acquires at least one light interference signal of a reflected light beam reflected from a cornea and a reference light beam. The data processing unit is electrically coupled with the optical module. The at least one light interference signal is transmitted to the data processing unit which determines an intraocular pressure detection area according to the at least one light interference signal. The data processing unit computes an intraocular pressure according to the at least one light interference signal acquired by the optical module.

In order to achieve the above-mentioned objects, the present invention provides an intraocular pressure detecting method, including the steps of: transmitting a light beam to an eyeball; acquiring at least one light interference signal of a reflected light beam of the light beam reflected from a cornea and a reference light beam; analyzing the at least one light interference signal to determine an intraocular pressure detection area; analyzing the light interference signal acquired; and computing the intraocular pressure.

### BRIEF DESCRIPTION OF THE DRAWINGS

FIG. 1 is a schematic diagram of an embodiment of the intraocular pressure detecting device of the present invention;

FIG. 2 is a schematic view of an embodiment of the intraocular pressure detecting device of the present invention;

FIG. 3 is a schematic views of an embodiment of the data processing unit determining the intraocular pressure detection area of the present invention;

FIG. 4 is a schematic diagram of another embodiment of the intraocular pressure detecting device of the present invention;

FIG. 5 is schematic view of another embodiment of the intraocular pressure detecting device of the present invention;

FIG. 6 is a graph of the pressure of the pressure wave versus the light interference signal;

FIG. 7 is a flow chart of an embodiment of the method for detecting the intraocular pressure of the present invention; and

FIG. 8 is a flow chart of another embodiment of the method for detecting the intraocular pressure of the present invention.

### DETAILED DESCRIPTION OF THE PREFERRED EMBODIMENT

The embodiments of the present invention are elaborated in coordination with the drawings in the following. In the specification, “embodiment”, “exemplified embodiment”, “a variety of embodiments”, etc. mean the specific characters, structures, or features, which are related to and/or included in the embodiments of the present invention. In the specification, the phrase “in the embodiment”, which appears everywhere, does not necessarily indicate the same embodiment. In the specification, technical terms such as “comparing”, “processing”, “computing”, “determining”, “recording”, “ordering” or the analogs thereof indicate the action or process of computer, computer system, or similar electronic calculator device. A physical quantity of a data (e.g., electron) in a register or memory in the computer system is operated or changed by the above-mentioned computer, computer system, or similar electronic calculator device to turn into a physical quantity of other data in a memory, a register of a computer system, or other information reservoir, transmitting or display device.

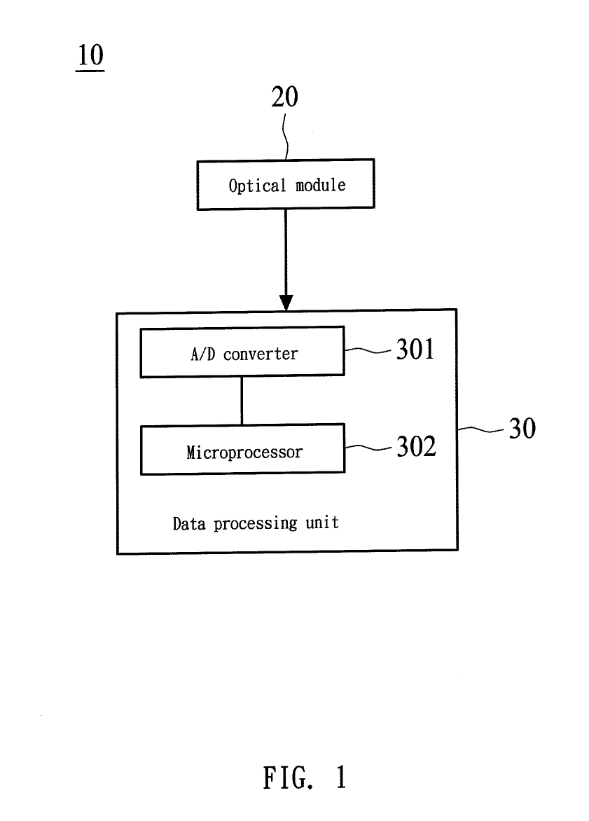

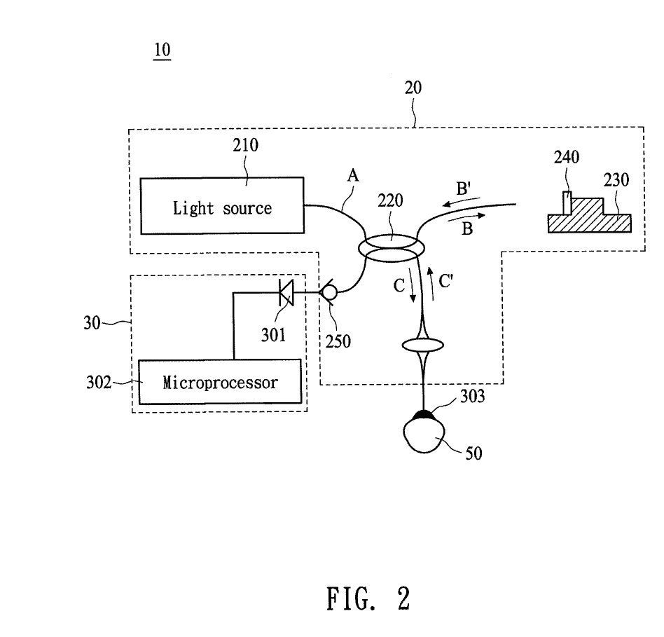

Please refer to an intraocular pressure detecting device 10 shown in FIG. 1. The intraocular pressure detecting device 10 includes an optical module 20 and a data processing unit 30. As FIGS. 1 and 2 show, the optical module 20 of the intraocular pressure detecting device 10 is preferably the Michelson interferometer but is not limited to the time domain optical coherence tomography. The frequency domain optical coherence tomography, the spatially encoded frequency domain optical coherence tomography, and the time encoded frequency domain optical coherence tomography can also be used in accordance with deferent design requirements of the device.

As FIGS. 1 and 2 shows, in the exemplified embodiment, the optical module 20 is embodied as the Michelson Interferometer. In the embodiment shown in FIGS. 1 and 2, the optical module 20 of the intraocular pressure detecting device 10 includes a light source 210, a coupler 220, a reflection platform 230, a reflective mirror 240, and a light sensor 250. The light source 210 of the optical module 20 of the intraocular pressure detecting device 10 emits a coherent light beam A; the light beam A is split into two light beams by the coupler 220 (e.g. a beam splitter); the two light beams are a first light beam B and a second light beam C, respectively. The first light beam B is transmitted to the reflection platform 230 which serves as a reference and is reflected from the reflective mirror 240. Meanwhile, the second light beam C is transmitted to a sample (e.g. the eyeball 50 in the embodiment) and is reflected from the sample (e.g., the cornea, the crystalline lens, or other objects to be tested of the eyeball 50). The first light beam B and the second light beam C are reflected as a light beam B′ and a light beam C′, respectively. Since the light beam C′ is reflected from the eyeball 50, the reflected light beam C′ has a time delay in comparison to the reflected light beam B′ (also referring to the optical path difference between the light beam C′ and the light beam B′). The reflected light beams B′ and C′ are interfered at the coupler 220 and then transmitted to the light sensor 250. In fact, the light sensor 250 can be a spectrometer, optical lens set, or other devices having the light-sensing function, but is not limited thereto. As a result, the light sensor 250 generates at least one light interference signal resulted from the interference between the reflected light beams B and C′.

As FIGS. 1 and 2 show, the light interference signal is transmitted to the data processing unit 30, where an A/D converter 301 converts the light interference signal from an analog light interference signal to a digital signal. The digital signal is transmitted to a microprocessor 302. The microprocessor 302 computes an optical data of the vertical cross section of the object to be tested based on the processing of the digital data about the light path difference between the reflected light beams B′ and C′.

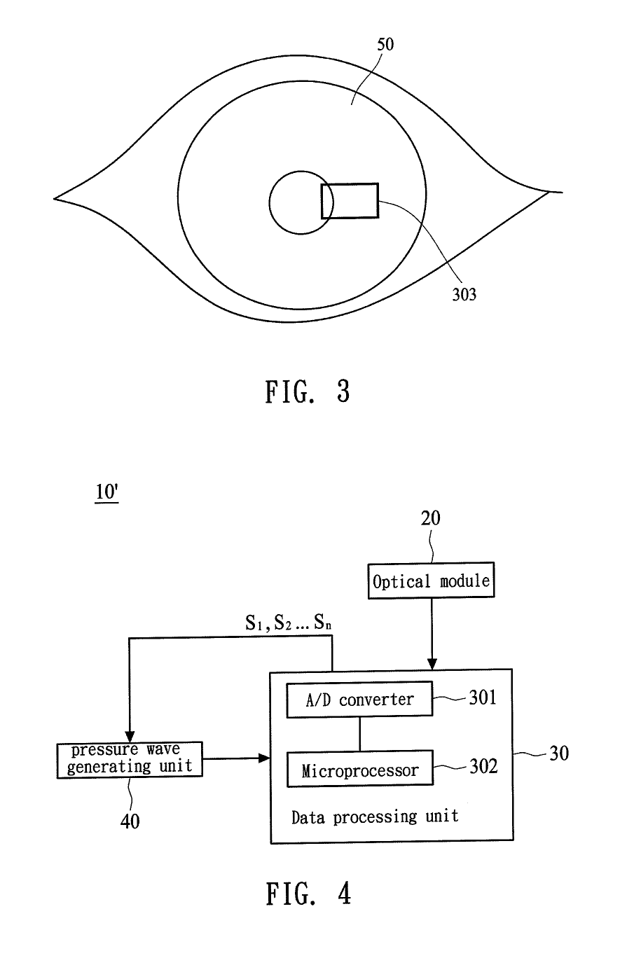

The data processing unit 30 can further compare the above-mentioned optical data of the vertical cross section of the object to be tested and use a preset intraocular pressure detection area to determine an intraocular pressure detection area 303 of the eyeball 50 as shown in FIG. 3. In brief, by means of the data processing unit 30 being electrically coupled with the optical module 20, the data processing unit 30 can determine the intraocular pressure detection area 303 according to the light interference signal.

In addition, if the optical module 20 is the above-mentioned optical interferometer, though the light interference signal generated by the optical interferometer can be provided to determine the intraocular pressure detection area 303 by the data processing unit 30, the above-mentioned light interference signal can also be analyzed and processed to determine an eyeball high-frequency oscillation of the eyeball 50 on witch the light beam C is incident in order to determine an initial intraocular pressure of the intraocular pressure detection area 303. However, such a method of measuring the intraocular pressure has a low signal-to-noise ratio (S/N) and a greater error; meanwhile, it requires a large amount of graph calculation and takes more time.

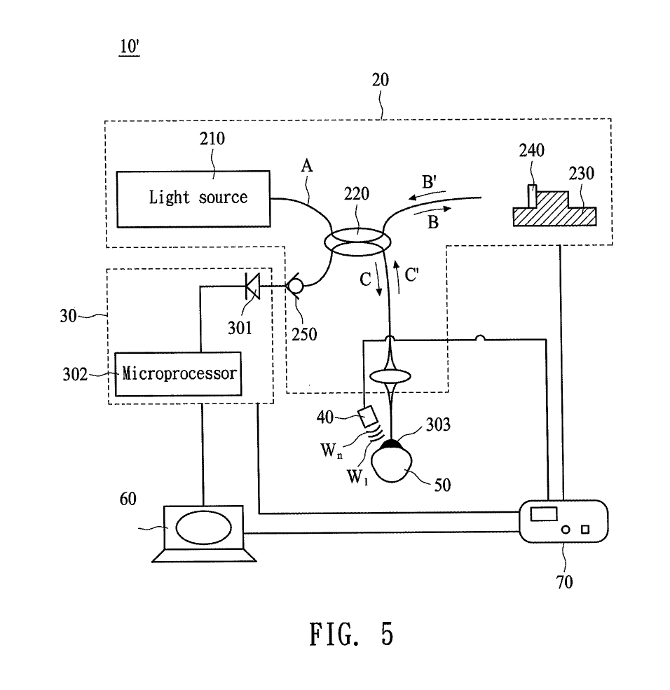

FIGS. 4 and 5 show an intraocular pressure detecting device 10′ of another embodiment of the present invention. The intraocular pressure detecting device 10′ further includes a pressure wave generating unit 40, a displaying unit 60, a control unit 70. The optical module 20 is already described in the above embodiment and not elaborated hereinafter. The control unit 70 is electrically coupled with the optical module 20, the data processing unit 30, the pressure wave generating unit 40, and the displaying unit 60, which can be individually or simultaneously controlled by the control unit 70.

Referring to FIG. 4, the data processing unit 30 is electrically coupled to the optical module 20, wherein the data processing unit 30 includes the ND converter 301 and the microprocessor 302. The light interference signal acquired by the optical module 20 includes, but is not limited to, corneal thickness data, corneal cross-sectional image data, and corneal curvature data. Practically, the cross-sectional image of the eyeball 50 is a light interference signal and can be transmitted to the ND converter 301 of the data processing unit 30 for further processing to generate an eyeball image signal (which is an electrical signal). The microprocessor 302 can compare and analyze the eyeball image signal to obtain a corneal pressure distribution and further determine an adequate intraocular pressure detection area 303, as FIG. 3 shows. In brief, the data processing unit 30 determines the intraocular pressure detection area 303 according to the eyeball image signal. The displaying unit 60 can further display the cross-sectional image of the object to be tested (e.g., the cornea) and information about the light interference signal. By comparing the corneal thickness data, the corneal cross-sectional image data, and the corneal curvature data through the high-frequency oscillation of the eyeball 50, the initial intraocular pressure of the intraocular pressure detection area 303 can be further determined.

The determination of the intraocular pressure detection area 303 is very important in measuring the intraocular pressure in practice, since the corneal curvature and the corneal thickness can influence the intraocular pressure measurement, resulting in a deviation of the value of the real intraocular pressure. In order to measure the intraocular pressure precisely and gain an more accurate value, after the determination of the intraocular pressure detection area 303, the pressure wave generating unit 40 performs the intraocular pressure measurement on the determined intraocular pressure detection area 303.

As FIG. 4 shows, the pressure wave generating unit 40 is electrically coupled with the data processing unit 30. The data processing unit 30 outputs a plurality of pressure wave-generating signals S₁, S₂ . . . Sₙ according to a time sequence to command the pressure wave generating unit 40 to generate a plurality of pressure waves W₁, W₂ . . . Wₙ according to the time sequence, as shown in FIG. 5. After these pressure waves W₁, W₂ . . . Wₙ imposing on the intraocular pressure area 303, the data processing unit 30 uses the above-mentioned light interference signals acquired by the optical module 20 to compute the intraocular pressure. The pressure waves generated by the pressure wave generating unit 40 can be selected from gas jet longitudinal waves, light waves, and ultrasonic waves. That is, the pressure wave generating unit 40 can be an air gun, a light pressure device, or an ultrasonic generator according to the corresponding pressure wave.

Practically, as FIGS. 4 and 5 show, when the data processing unit 30 outputs a plurality of pressure wave-generating signals S₁, S₂ . . . Sₙ according to a time sequence to command the pressure wave generating unit 40 to generate a plurality of pressure waves W₁, W₂ . . . Wₙ, the pressure imposed by the pressure waves W₁, W₂ . . . Wₙ preferably but not necessary increases according to the time sequence. In other embodiments, the pressure imposed by the pressure waves W₁, W₂ . . . Wₙ can be constant. As the pressure wave generating unit 40 generates the pressure waves W₁, W₂ . . . Wₙ, the pressure wave generating unit 40 simultaneously transmits the pressure value of the pressure waves to the data processing unit 30. Since the eyeball 50 has the intraocular pressure, if the pressure of the pressure wave is less than or equal to the intraocular pressure, the eyeball 50 will not deform when the pressure wave imposes on the eyeball 50. However, if the pressure of the pressure wave is greater than the intraocular pressure, the eyeball 50 will deform and the amount of deformation is dependent from the magnitude of the pressure of the pressure wave. When the pressure wave generating unit 40 causes the deformation of the eyeball, the optical module 20 can acquire a plurality of light interference signals (e.g., I₁, I₂) at different points of time and elevate the signal-to-noise ratio of the intraocular pressure measurement by cross comparison described in the following.

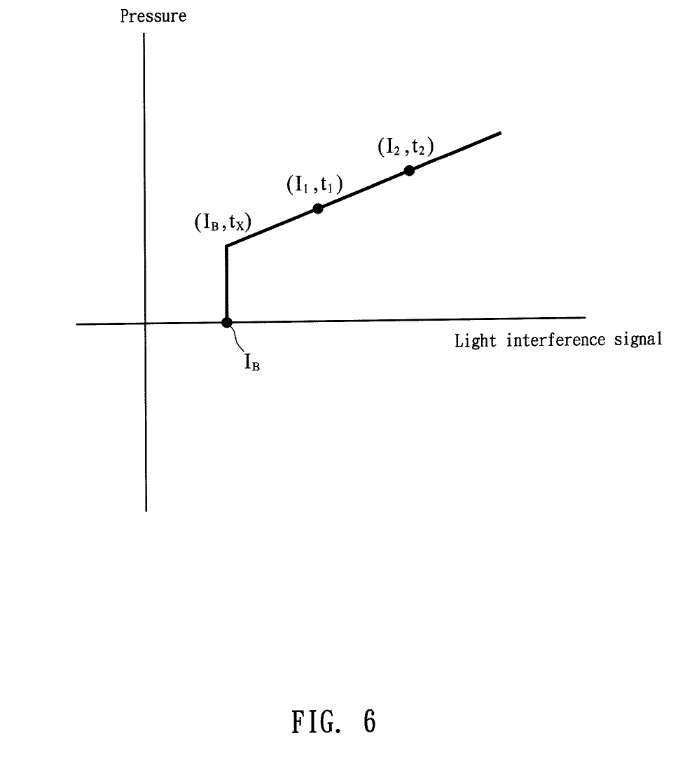

As FIGS. 4, 5, and 6 show, the optical module 20 will acquire an initial light interference signal I_{B} before the pressure wave is imposed. After the pressure waves W₁, W₂ . . . Wₙ are imposed, the optical module 20 will acquire a first light interference signal I₁ at a first point of time t₁ and a second light interference signal I₂ at a second point of time t₂. The two points (I₁, t₁), (I₂, t₂) can determine a linear equation to compute a time tₓ of the light interference signal I_{B}. The pressure value at the time tₓ, which is the intraocular pressure, can be deduced from the pressure value transmitted from the pressure wave generating unit 40 to the data processing unit 30 at the time tₓ. In other words, the data processing unit 30 can determine the intraocular pressure according to the extrapolation or the interpolation of the first light interference signal I₁ and the second light interference signal I₂ as well as the initial light interference signal I_{B}. The deduced value of the intraocular pressure can be used to calibrate the initial intraocular pressure computed by the optical module 20 (e.g. optical coherence tomography) using the eyeball high-frequency oscillation so that the signal-to-noise ratio is elevated and the error is reduced. Meanwhile, the corneal curvature data and the corneal thickness data acquired by the optical module 20 can be used to calibrate the value of the intraocular pressure.

In addition to the computation of the intraocular pressure by means of the reflected light beams C′, B′, the intraocular pressure can also be computed by using the cross-sectional image of the eyeball 50. Therefore, the precision of the computed intraocular pressure is improved. In addition, the corneal curvature and the corneal thickness obtained from the cross-sectional image of the eyeball 50 can also be used to calibrate the possible error caused by the corneal curvature and the corneal thickness.

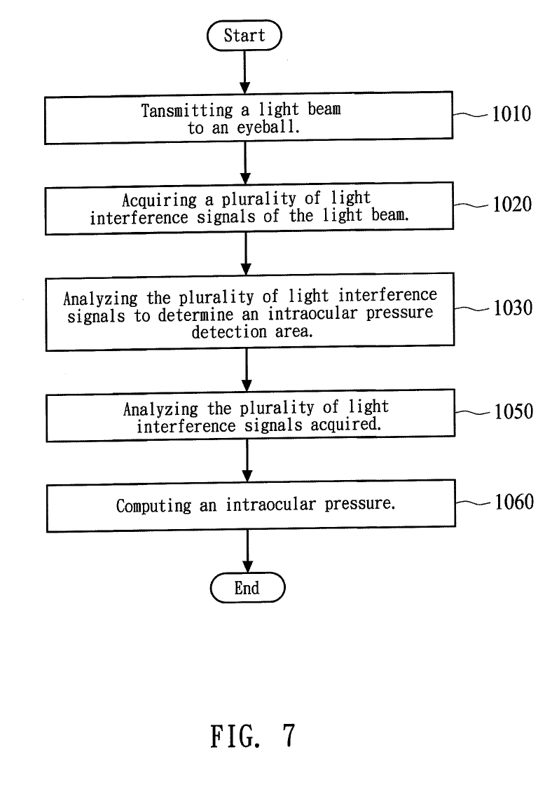

FIG. 7 shows a method for detecting an intraocular pressure, comprising the following steps: a step 1010 of transmitting the light beam to the eyeball; a step 1020 of acquiring a plurality of light interference signals of the light beam; a stop 1030 of analyzing the plurality of light interference signals to determine the intraocular pressure detection area; a step 1050 of analyzing the plurality of light interference signals acquired; and a step 1060 of computing the intraocular pressure.

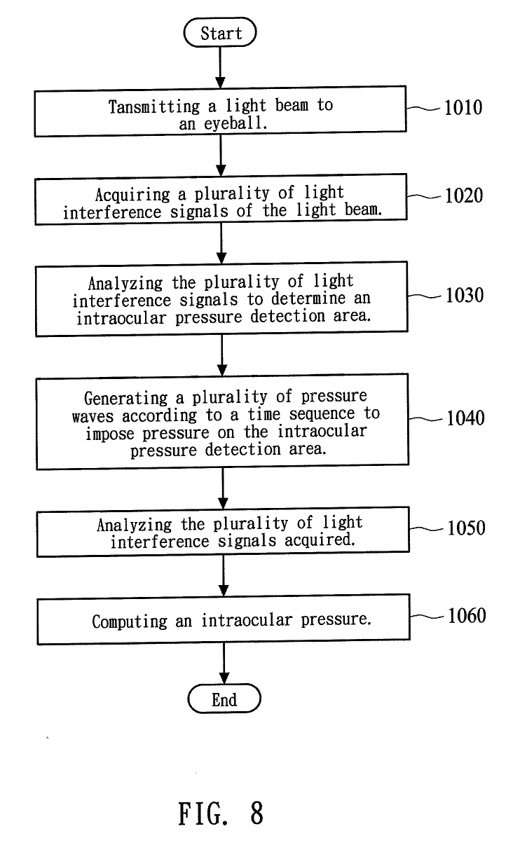

Another method for detecting the intraocular pressure as shown in FIG. 8 further includes a step 1040 of generating a plurality of pressure waves according to a time sequence to impose pressure on the intraocular pressure detection area in addition to the steps 1010, 1020, 1030, 1040, 1050, and 1060 shown in FIG. 7.

Although the preferred embodiments of present invention have been described herein, the above description is merely illustrative. The preferred embodiments disclosed will not limited the scope of the present invention. Further modification of the invention herein disclosed will occur to those skilled in the respective arts and all such modifications are deemed to be within the scope of the invention as defined by the appended claims.

What is claimed is:

1. An intraocular pressure detecting device, comprising:
an optical module comprising a coupler, wherein the optical module transmits a light beam to the coupler to generate a first light beam and a second light beam, the second light beam is transmitted to an eyeball, the optical module acquires at least one light interference signal resulted from an interference between a reflected light beam of the second light beam reflected from a cornea and a reflected light beam of the first light beam; and
a data processing unit electrically coupled with the optical module, wherein the data processing unit determines an intraocular pressure detection area according to the at least one light interference signal, and the data processing unit computes an intraocular pressure according to the at least one light interference signal acquired by the optical module.

2. The intraocular pressure detecting device of claim 1, further comprising a pressure wave generating unit electrically coupled with the data processing unit, wherein the data processing unit commands the pressure wave generating unit to generate a plurality of pressure waves according to a time sequence; after the pressure waves impose pressure on the intraocular pressure detection area, the data processing unit computes the intraocular pressure according to the at least one light interference signal acquired by the optical module.

3. The intraocular pressure detecting device of claim 1, wherein the pressure waves are selected from a gas jet longitudinal wave, a light wave, and an ultrasonic wave.

4. The intraocular pressure detecting device of claim 2, wherein before the pressure waves impose pressure on the intraocular pressure detection area, the optical module acquires an initial light interference signal.

5. The intraocular pressure detecting device of claim 4, wherein after the pressure waves impose pressure on the intraocular pressure detection area, the optical module acquires a first light interference signal at a first point of time and acquires a second light interference signal at a second point of time, the data processing unit determines the intraocular pressure according to the initial interference signal and an extrapolation or an interpolation of the first light interference signal and the second light interference signal.

6. The intraocular pressure detecting device of claim 1, wherein the optical module further comprises a light source, a reflection platform, a reflective mirror, and a light sensor, the light source generates the light beam, the first light beam is transmitted to and reflected from the reflective mirror on the reflection platform, and the light sensor detects the interference to generate the at least one light intraocular signal.

7. A method for detecting an intraocular pressure, comprising:
transmitting a light beam to an eyeball;
acquiring at least one light interference signal of the light beam;
determining an intraocular pressure detection area according to the at least one light interference signal;
analyzing the at least one light interference signal acquired; and
computing an intraocular pressure.

8. The method of claim 7, further comprising:
generating a plurality of pressure waves according to a time sequence to impose pressure on the intraocular pressure detection area.

9. The method of claim 8, further comprising:
acquiring an initial light interference signal before the pressure waves impose pressure on the intraocular pressure detection area.

10. The method of claim 9, wherein the computing step of the intraocular pressure comprises:
after the pressure waves impose pressure on the intraocular pressure detection area, acquiring a first light interference signal at a first point of time and acquiring a second light interference signal at a second point of time, and determining the intraocular pressure according to the initial interference signal and an extrapolation or an interpolation of the first light interference signal and the second light interference signal.
